(19) United States
(12) Patent Application Publication
(10) Pub. No.: US 2024/0100257 A1
(43) Pub. Date: Mar. 28, 2024

(54) PLUNGERS, PLUNGER ASSEMBLIES, SYRINGES, AND METHODS OF MAKING AND USING SAME

(71) Applicant: SiO2 Medical Products, Inc., Auburn, AL (US)

(72) Inventors: Javier Ferrer, Auburn, AL (US); Brian Russell Lilly, Auburn, AL (US)

(21) Appl. No.: 18/512,258

(22) Filed: Nov. 17, 2023

Related U.S. Application Data
(63) Continuation of application No. PCT/US2022/072499, filed on May 23, 2022.
(60) Provisional application No. 63/191,909, filed on May 21, 2021.

(51) Int. Cl. A61M 5/315

(52) U.S. Cl. CPC A61M 5/31515; A61M 5/31505; A61M 2205/0238

(57) ABSTRACT

Plunger assemblies (20) including a plunger sleeve (34), a plunger rod (22), and an axial protrusion (30) disposed within an inner cavity (40) of the plunger sleeve. Application of a distal force onto the plunger via the plunger rod causes the axial protrusion to contact and apply pressure to an engagement surface (50) in the inner cavity. The engagement surface is configured to receive distal force from the end of the axial protrusion. This causes the plunger to elongate and slightly constrict, thus reducing break loose force and facilitating transition from storage mode to dispensing mode of the plunger.

### CROSS-REFERENCE TO RELATED APPLICATION

The present application claims priority to U.S. Provisional Application No. 63/191,909, filed May 21, 2021 and titled “PLUNGERS, PLUNGER ASSEMBLIES AND SYRINGES”, the subject matter of which is hereby incorporated by reference.

### BACKGROUND

### 1. Field

The disclosed concept relates to plungers and their use in drug delivery devices, such as pre-filled, filled before use, and or empty syringes, cartridges, or auto-injectors.

### 2. Description of Related Art

The present disclosure predominantly describes use of plungers and plunger assemblies according to the disclosed concept in connection with pre-filled syringes. However, the presently disclosed technology is not limited to pre-filled syringes, but may include other drug delivery devices, such as pre-filled, filled before use, or empty syringes, cartridges, and auto-injectors.

Pre-filled parenteral containers, such as syringes or cartridges, are commonly prepared and sold so that the syringe does not need to be filled by the patient or caregiver before use. The syringe, and more specifically the barrel of the syringe, may be prefilled with a variety of different injection products, including, for example, saline solution, a dye for injection, or a pharmaceutically active preparation, among other items. This is particularly the case for syringes that are used to dispense very small and precise amounts of injectable product, such as for ophthalmic use.

Pre-filled parenteral containers are typically sealed with a rubber plunger, which provides closure integrity over the shelf life of the container's contents. To use the prefilled syringe, the packaging and cap are removed, optionally a hypodermic needle or another delivery conduit is attached to the dispensing end of the barrel, the delivery conduit or syringe is moved to a use position (such as by inserting it into a patient's blood vessel or into an apparatus to be rinsed with the contents of the syringe), and the plunger is advanced axially down the barrel to inject contents of the barrel to the point of application.

Seals provided by rubber plungers in the barrel typically involve the rubber of the plunger being pressed against the barrel. Typically, the rubber plunger is larger in diameter than the internal diameter of the barrel. Thus, to displace the rubber plunger when the injection product is to be dispensed from the syringe requires overcoming this pressing force of the rubber plunger. Not only does this pressing force provided by the rubber seal typically need to be overcome when initially moving the plunger, but this force also needs to continue to be overcome as the rubber plunger is displaced along the barrel during the dispensing of the injection product.

The need for even slightly elevated forces to advance the plunger in the syringe may increase the difficulty a user may have in dispensing the injection product from the syringe, which can be undesirable. Such elevated forces may also hinder a user's ability to dispense small and precise amounts, such as during a priming step with an ophthalmic syringe. Such elevated forces can prove particularly problematic for auto injection systems where the syringe is placed into the auto injection device and the plunger is advanced by a fixed spring.

Accordingly, primary considerations concerning the use of a plunger in a pre-filled parenteral container include: (1) adequacy of the seal provided by the plunger within the container during storage and use, for example whether the plunger provides container closure integrity (“CCI”, defined below); and (2) plunger force (defined below) required to dispense syringe contents.

In practice, CCI and plunger force tend to be competing considerations. In other words, absent other factors, the tighter the fit between the plunger and the interior surface of the container to maintain adequate CCI, the greater the force necessary to advance the plunger in use. In the field of medical syringes, it is important to ensure that the plunger can move at a substantially constant speed and with a substantially constant force when advanced in the barrel. In addition, the force necessary to initiate plunger movement and then continue advancement of the plunger should be low enough to enable precise administration by a user and comfort for a patient.

Plunger force is essentially a function of the coefficients of friction of each of the contacting surfaces (e.g., the plunger surface and interior syringe wall surface) and the normal force exerted by the plunger against the interior wall of the syringe. The greater the respective coefficients of friction and the greater the normal force, the more force required to advance the plunger. Accordingly, efforts to improve plunger force should be directed to reducing friction and lowering normal force between contacting surfaces. However, such efforts should be tempered by the need to maintain an adequate seal, e.g., CCI, as discussed above.

To reduce friction and thus improve plunger force, lubrication may be applied to the plunger, the interior surface of the container, or both. Liquid or gel-like flowable lubricants, such as free silicone oil (e.g., polydimethylsiloxane or “PDMS”), may provide a desired level of lubrication to optimize plunger force. Flowable lubricants, when used with pre-filled syringes, may migrate away from the plunger over time, resulting in spots between the plunger and the interior surface of the container with little or no lubrication. This may cause a phenomenon known as “sticktion”, an industry term for the adhesion between the plunger and the barrel that needs to be overcome to break out the plunger and allow it to begin moving.

### SUMMARY

There is a need for optimizing plunger force in a parenteral container while maintaining adequate CCI to prevent drug leakage, protect the drug product and attain sufficient product shelf life.

A coupling pin of conventional geometry is shown as element 130a in FIGS. 3A and 3B of WO 2019/199901, although the telescoping configuration of the rod itself in that publication is not conventional. Applicant has found that when inserting a plunger rod having a coupling pin of conventional geometry into a plunger in a prefilled syringe, the plunger moves in a distal direction, which is undesirable when coupling a plunger rod to a plunger during or after a filling operation.

There is a need for a plunger rod having a plunger coupling pin or other structure configured to minimize insertion force when assembling the plunger rod to the plunger while simultaneously ensuring that the plunger rod does not detach from the plunger too easily if drawn in a proximal direction (i.e., away from the drug product). There is a need for these features in addition to a plunger rod that is configured for gas sterilization. e.g., ethylene oxide sterilization.

The presently disclosed technology overcomes the above and other drawbacks of the prior art.

Accordingly, in one optional embodiment, a plunger assembly for use in a medical barrel can include a plunger rod having a distal end and a proximal end. An axial protrusion can be secured to or extends from the distal end of the plunger rod. The axial protrusion can include a stem portion having an essentially uniform cross section and is optionally generally cylindrical. The stem portion can lead to a head portion. The head portion can have a proximal end with a greater cross-sectional width or diameter than that of the stem portion. The head portion can have a distal end with a rounded tip. The head portion can have a symmetrical cross-section with an outer contour that flares radially outward in a proximal direction. At least a portion of the outer contour optionally has a curved surface including a radius with an imaginary center located outside of the head portion. The outer contour terminating an outer edge at the proximal end of the head portion. A shelf of the head portion can extend radially inward from the outer edge and terminates at the stem portion. The plunger assembly can further include a plunger having a plunger sleeve with an exterior surface and an interior surface surrounding an inner cavity. The exterior surface can include a distal nose cone and an outer annular wall extending proximally from the nose cone and leading to an opening at a proximal end of the plunger sleeve. The opening can receive the axial protrusion such that the axial protrusion extends into the inner cavity and contacts an engagement surface of the interior surface. The engagement surface can be configured to receive a force applied in a distal direction by the axial protrusion to move the plunger assembly in a distal direction when the plunger rod is moved in a distal direction. The distal end of the plunger rod does not initially contact the proximal end of the plunger sleeve when the plunger is in a pre-elongation state. The plunger rod and axial protrusion can be provided as a single piece, of unitary construction.

In an optional aspect, the disclosed concept relates to a prefilled syringe with the plunger of the aforementioned plunger assembly disposed within a medical barrel containing an injectable product. The plunger can be configured to provide sufficient CCI and gas-tight sealing over a desired shelf life when the plunger is in storage mode. The plunger can be converted to dispensing mode by axially elongating the plunger, which slightly constricts the outer annular wall of the plunger to reduce the plunger's radial compression against the barrel inner wall. This renders it easier to advance the plunger down the barrel, while still maintaining at least a liquid tight seal.

Optionally, in any embodiment, the axial protrusion and/or the interior surface of the plunger comprises a flowable lubricant, such as silicone oil. Optionally, in any embodiment, the axial protrusion and/or the interior surface of the plunger comprise a lubricity coating, optionally wherein the lubricity coating is a coating applied using plasma enhanced chemical vapor deposition (“PECVD”) having one of the following atomic ratios: SiwOxCy or SiwNxCy, where w is 1, x is from about 0.5 to 2.4 and y is from about 0.6 to about 3.

Optionally, in any embodiment, the plunger is made from a thermoplastic elastomer or rubber, optionally a bromobutyl rubber, optionally having a durometer of from 30 to 70, preferably from 40 to 60. Optionally, in any embodiment, the outer annular wall of the plunger includes at least one annular rib, optionally at least two annular ribs, optionally at least three annular ribs.

Optionally, in any embodiment, the disclosed concept relates to a syringe comprising a medical barrel with a plunger disposed therein, the plunger being a component of any embodiment of a plunger assembly described herein. Such a syringe is optionally a pre-filled syringe and can include an injectable product stored within a product containing area. In any embodiment, the plunger can include a stretch zone adapted to undergo elongation along a central axis of the plunger upon application of a force in the distal direction by the axial protrusion onto the engagement surface of the inner cavity of the plunger. Such elongation reduces and/or constricts an outer profile of the outer annular wall along the stretch zone. Optionally, the elongation of the plunger is less than 1.5 mm.

In any syringe embodiment, the plunger rod does not initially contact the plunger sleeve when the plunger is in the pre-elongation state. Once the plunger is transitioned to dispensing mode, wherein the plunger undergoes elongation and displacement down the barrel, in some embodiments the plunger does not contact the plunger sleeve while in other embodiments it does.

In any embodiment, elongation of the plunger constricts the outer annular wall along the stretch zone, thereby reducing radial compression of the outer annular wall against the inner wall of the medical barrel.

Optionally, in any embodiment, the engagement surface is provided on a distal section of the interior surface of the inner cavity of the plunger and a distal portion of the axial protrusion, optionally solely the distal portion of the axial protrusion, contacts the engagement section.

The plunger is configured to be translated in both distal and proximal directions by the plunger rod when the plunger is disposed in a prefilled syringe.

Optionally, in any embodiment of a pre-filled syringe, when in storage mode, the plunger exerts outward radial compression against the inner wall of the medical barrel to form a liquid tight, CCI, and gas-tight interface therewith. After the plunger is converted to dispensing mode, it continues to maintain a liquid tight interface and optionally maintains a CCI and gas-tight interface as the plunger is advanced down the barrel to dispense an injectable product.

Optionally, in any embodiment of a pre-filled syringe, flowable lubricant, such as silicone oil, is coated onto the syringe sidewall and/or the outer annular wall of the plunger. Optionally, in an alternative embodiment, no flowable lubricant is provided between the plunger and the syringe sidewall.

Optionally, in any embodiment of a pre-filled syringe, break loose force of the plunger is at or below 15 N, optionally below 10 N, optionally below 9 N, optionally below 8 N, optionally below 7 N, optionally below 6 N, optionally from 4 to 8 N, optionally from 4 to 6 N. Optionally, this break loose force is achieved without a flowable lubricant between the plunger and the syringe sidewall. Optionally, in any embodiment of a prefilled syringe, the differential between break loose force and glide force is below 6 N, optionally below 4 N, optionally below 3 N, optionally below 2 N, optionally below 1.5 N, optionally below 1.0 N, optionally below 0.5 N, optionally below 0.25 N, optionally from 0.5 N to 4 N.

Optionally, in any embodiment, the plunger can include a fluoropolymer film coating applied on its outer surface. This can provide a drug contacting surface and can optionally extend along at least a portion of the outer annular wall of the plunger to provide lubricity to the plunger to reduce plunger force.

Optionally, in any embodiment, four weeks post stoppering, the plunger does not move distally or moves minimally distally when the plunger rod is inserted into the plunger. Optionally, in any embodiment, once the plunger rod is inserted into the plunger, a proximal force of greater than 15 N, optionally greater than 10 N, optionally greater than 5 N, optionally greater than 3.5 N, optionally greater than 3 N, applied to the plunger rod, is required to decouple the plunger rod from the plunger when the plunger is disposed in the syringe barrel.

Optionally, in any embodiment, the plunger rod is injection molded as a single component.

Optionally, in any embodiment, the plunger rod is retained by the plunger after removing the closure and affixing a needle (e.g., 30-gague needle) and moving the plunger to the dose line position (priming). After priming, the plunger rod remains coupled so that the plunger moves before it decouples when the plunger rod is drawn back in the proximal direction.

Optionally, in any embodiment, the plunger rod coupling pin couples and mates with the cavity of a West 2432 4023/50 0.5 mL syringe stopper.

Optionally, in any embodiment, the plunger does not break CCI during assembly, transport, and/or use.

Optionally, in any embodiment, the plunger rod is substantially free of any solid standing pad or valve gate. Optionally, in any embodiment, the plunger rod is substantially free of any gate stringers. Optionally, in any embodiment, the plunger rod is substantially free of any parting-line mismatch or (lash.

Optionally, in any embodiment, the plunger rod is suitable for gas sterilization, e.g., ethylene oxide sterilization.

Optionally, in any embodiment, the plunger rod remains intact during intended use conditions of the final product.

Optionally, the plunger rod is suitable for automated or semi-automated assembling operations.

Optionally, plunger rod insertion into the plunger must minimize plunger movement.

Optionally, a syringe assembly with the plunger rod and plunger described herein provides a minimum shelf life of 36 months.

Optionally, the plunger rod is made from medical grade polypropylene.

### BRIEF DESCRIPTION OF SEVERAL VIEWS OF THE DRAWINGS

The foregoing summary, as well as the following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is an isometric view of an exemplary pre-filled syringe assembly with which the disclosed concept may be implemented.

FIG. 2 is an isolated axial sectional view of an exemplary plunger that may be used according to any embodiment of the disclosed concept.

FIG. 3 is an isometric view of an exemplary one-piece plunger rod and axial protrusion assembly in accordance with a first optional embodiment of the disclosed concept.

FIG. 4 is a top view of the plunger rod and axial protrusion assembly of FIG. 3.

FIG. 5 is a side view of the plunger rod and axial protrusion assembly of FIG. 3.

FIG. 6A is a magnified isolated side view of the axial protrusion of the plunger rod and axial protrusion assembly of FIG. 3.

FIG. 6B is a perspective view of the axial protrusion of the plunger rod and axial protrusion assembly shown in FIG. 6A.

FIG. 6C is a front elevation view of the axial protrusion of the plunger rod and axial protrusion assembly of FIG. 6A.

FIG. 7 is a magnified partial sectional view of the plunger rod and axial protrusion assembly of FIG. 6A.

FIG. 8A is an axial sectional view of a partial syringe assembly comprising the one-piece plunger rod of FIG. 3 with an axial protrusion inserted into a plunger within the syringe barrel.

FIG. 8B is an axial sectional view of the partial syringe assembly of FIG. 8A, prior to insertion of the axial protrusion into the plunger.

FIG. 9A is an enlarged sectional view of a first alternative embodiment of the inner surface of the syringe of FIG. 8A, including a tri-layer coating set disposed thereon.

FIG. 9B is an enlarged section view of a second alternative embodiment of the inner surface of the syringe of FIG. 8A, including a four layer coating set disposed thereon.

FIG. 9C is an enlarged sectional view of a third alternative embodiment of the inner surface of the syringe of FIG. 8A, including an organo-siloxane coating disposed thereon.

FIG. 10A is partial axial sectional view of the partial syringe assembly of FIG. 5 attached to the plunger of FIG. 2 in a first configuration or storage mode.

FIG. 10B is partial axial sectional view of the partial syringe assembly of FIG. 5 attached to the plunger of FIG. 2 in a second configuration.

FIG. 10C is partial axial sectional view of the partial syringe assembly of FIG. 5 attached to the plunger of FIG. 2 in a third configuration or dispensing mode, illustrating stretching of the plunger to transition from storage mode to dispensing mode.

FIG. 11 is a cross sectional view of an optional embodiment of a plunger rod axial protrusion inserted into and connecting with the inner cavity of a plunger.

### DETAILED DESCRIPTION

The disclosed concept will now be described more fully with reference to the accompanying drawings, in which several embodiments are shown. The presently disclosed technology may, however, be embodied in many different forms and should not be construed as limited to the embodiments set forth here. Rather, these embodiments are examples of the presently disclosed technology, which has the full scope indicated by the language of the claims. Like numbers refer to like elements throughout. Unless indicated otherwise, the features characterizing the embodiments and aspects described in the following may be combined with each other, and the resulting combinations are also embodiments of the presently disclosed technology.

### Definitions

As used in this disclosure, an “organosilicon precursor” is a compound having at least one of the linkages:

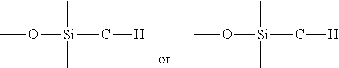

which is a tetravalent silicon atom connected to an oxygen or nitrogen atom and an organic carbon atom (an organic carbon atom being a carbon atom bonded to at least one hydrogen atom). A volatile organosilicon precursor, defined as such a precursor that can be supplied as a vapor in a plasma enhanced chemical vapor deposition (PECVD) apparatus, is an optional organosilicon precursor. Optionally, the organosilicon precursor is selected from the group consisting of a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, and a combination of any two or more of these precursors. Preferably, the organosilicon precursor is octamethylcyclotetrasiloxane (OMCTS). Values of w, x, y, and z are applicable to the empirical composition Si_{w}OₓC_{y}H_{z} throughout this specification. The values of w, x, y, and z used throughout this specification should be understood as ratios or an empirical formula (for example for a coating or layer), rather than as a limit on the number or type of atoms in a molecule. For example, octamethylcyclotetrasiloxane, which has the molecular composition Si₄O₄C₈H₂₄, can be described by the following empirical formula, arrived at by dividing each of w, x, y, and z in the molecular formula by 4, the largest common factor: Si₁O₁C₂H₆. The values of w, x, y, and z are also not limited to integers. For example, (acyclic) octamethyltrisiloxane, molecular composition Si₃O₂C₈H₂₄, is reducible to Si₁O_{0.67}C_{2.67}H₈. Also, although SiOₓC_{y}H_{z} is described as equivalent to SiOₓC_{y}, it is not necessary to show the presence of hydrogen in any proportion to show the presence of SiOₓC_{y}.

“Container closure integrity” or “CCI” refers to the ability of a container closure system, e.g., a plunger disposed in a prefilled syringe barrel, to provide protection and maintain efficacy and sterility during the shelf life of a sterile product contained in the container.

The “plunger sliding force” (synonym to “glide force”, “maintenance force”, or Fₘ, also used in this description) in the context of the presently disclosed technology is the force required to maintain movement of a plunger tip in a syringe barrel, for example during aspiration or dispense. It can advantageously be determined using the ISO 7886-1:1993 test known in the art. A synonym for “plunger sliding force” often used in the art is “plunger force” or “pushing force.”

The “plunger breakout force” (synonym to “breakout force”. “break loose force”, “initiation force”, Fᵢ, also used in this description) in the context of the presently disclosed technology is the initial force required to initiate movement of the plunger in a syringe, for example in a prefilled syringe.

The term “syringe” is to be understood broadly and includes cartridges, injection “pens,” and other types of barrels or reservoirs adapted to be assembled with one or more other components to provide a functional syringe. “Syringe” also includes related articles or devices, such as auto-injectors, which provide a mechanism for dispensing the contents. Optionally, “syringe” may include prefilled syringes. A “syringe” as used herein may also apply to vaccine dispensing syringes comprising a product space containing a vaccine. A “syringe” as used herein may also have applications in diagnostics, e.g., a sampling device comprising a medical barrel prefilled with a diagnostic agent (e.g., contrast dye) or the like.

“PECVD” refers to plasma enhanced chemical vapor deposition.

The terms “distal” and “proximal” are used throughout this specification. The terms “distal” and “proximal” refer generally to a spatial or positional relationship relative to a given reference point, wherein “proximal” is a location at or comparatively closer to that reference point and “distal” is a location further from that reference point.

“Proximal” and “distal” may also be used to refer to force vectors and direction of displacement. For example, the pushing force to dispense syringe contents would be applied in a “distal direction” or “distally,” i.e., a force pushing a plunger to advance it down toward the dispensing end or distal end of the medical barrel. By contrast, a pulling force on a plunger rod to pull it away from the dispensing end of the barrel would be a force applied in a “proximal direction” or “proximally.”

### Optional Syringe Barrel Materials

Optionally, syringes according to any embodiment of the presently disclosed technology can be made from one or more injection moldable thermoplastic materials including, but not limited to: an olefin polymer, polypropylene (PP); polyethylene (PE); cyclic olefin copolymer (COC); cyclic olefin polymer (COP); polymethylpentene; polyester, polyethylene terephthalate; polyethylene naphthalate: polybutylene terephthalate (PBT); PVdC (polyvinylidene chloride): polyvinyl chloride (PVC); polycarbonate: polymethylmethacrylate: polylactic acid; polylactic acid; polystyrene; hydrogenated polystyrene; poly(cyclohcxylethylene) (PCHE); nylon: polyurethane polyacrylonitrile; polyacrylonitrile (PAN); an ionomeric resin; Surlyn® ionomeric resin. For applications in which clear and glass-like polymers are desired (e.g., for syringes and vials), a cyclic olefin polymer (COP), cyclic olefin copolymer (COC) or polycarbonate may be preferred. Such materials may be manufactured. e.g., by injection molding or injection stretch blow molding, to very tight and precise tolerances (generally much tighter than achievable with glass). Alternatively, syringes according to embodiments of the presently disclosed technology can be made from glass.

Syringe and Plunger Assembly Components and Embodiments

As set forth above, the disclosed concept optionally relates to plungers that are convertible to a dispensing mode by actuating the plunger to stretch or elongate it, which helps facilitate low and smooth plunger force when dispensing syringe contents. Applicant SiO2 Medical Products. Inc. has developed other convertible plungers, which are described in some of its published international patent applications, including WO2015/054282, published Apr. 16, 2015, WO2016/039816, published Mar. 17, 2016, WO2017/011599, published Jan. 19, 2017, WO2017/209800, published Dec. 7, 2017 and WO2019/199901, published Oct. 17, 2019. Each of these published applications are incorporated by reference herein in their entireties for all that they disclose. While the convertible feature is preferred in many applications, it is not required in all embodiments of the disclosed concept.

In another aspect, the disclosed concept relates to plunger rod and plunger assemblies configured to facilitate connection of the plunger rod to the plunger while the plunger is disposed within a filled syringe and enabling retraction of the plunger when the plunger rod is pulled.

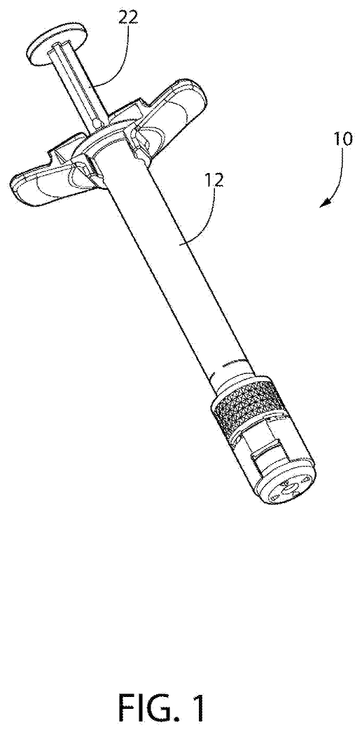

Referring to FIG. 1, there is shown an exemplary embodiment of a syringe assembly 10 (e.g., a prefilled syringe assembly) in accordance with an optional aspect of the disclosed concept. The syringe assembly includes a medical barrel 12 and a plunger assembly disposed at least partially therein, of which a portion of a plunger rod 22 is shown in FIG. 1. The syringe assembly 10 may include accoutrements typically included with prefilled syringes, such as an end cap, optionally a luer fitting to which a syringe needle may be secured at time of use, etc. Alternatively, the syringe may be a staked needle syringe.

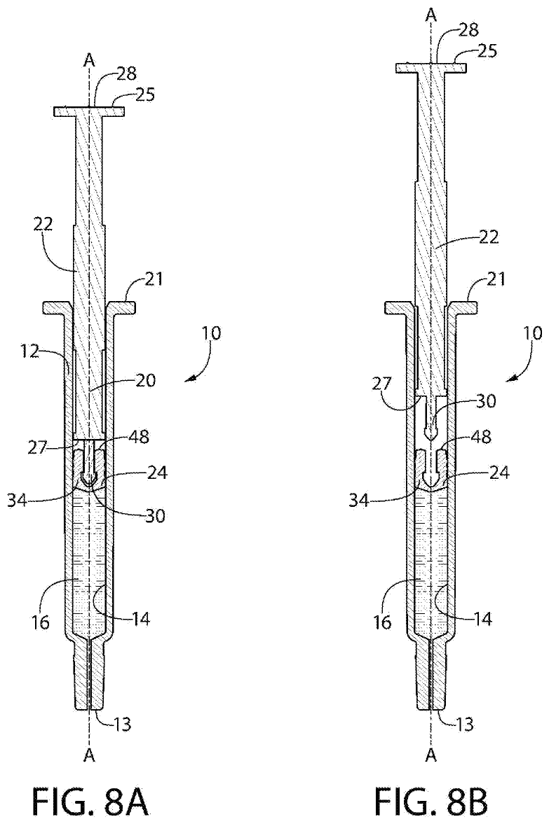

In any embodiment, for example as shown in FIGS. 8A and 8B, the syringe assembly 10 includes the hollow medical barrel 12 having a central longitudinal axis A. The medical barrel 12 has a cylindrical inner wall 14 and is configured to hold an injectable liquid 16, optionally a drug product, therein. The injectable liquid 16 is preferably prefilled to provide a prefilled syringe. In an alternative embodiment, the syringe is not prefilled. A needle (not shown) may be provided at the distal end of the medical barrel 12 to dispense the injectable liquid 16.

As applied herein to medical barrels, referring to FIGS. 8A and 8B, the relevant reference point is the back end of the barrel, for example, the flange 21 at the top of the barrel 12. The distal end is at the bottom or dispensing end 13 of the barrel 12, where a needle may be mounted. This same convention applies to other components described herein, such as plungers and plunger assemblies.

In any embodiment of a syringe assembly, or as aspect in and of itself of the disclosed concept, a plunger assembly 20 is provided and shown in FIGS. 8A and 8B. The plunger assembly 20 comprises a plunger rod 22, an axial protrusion 30 secured to or extending from (e.g., integral with) a distal end 27 of the plunger rod 22, and a plunger 24 into which the axial protrusion 30 is disposed. The distal end 27 can be a flat or planar surface that surrounds and/or extends radially outwardly with respect to the axial protrusion 30. This assembly is discussed in greater detail below.

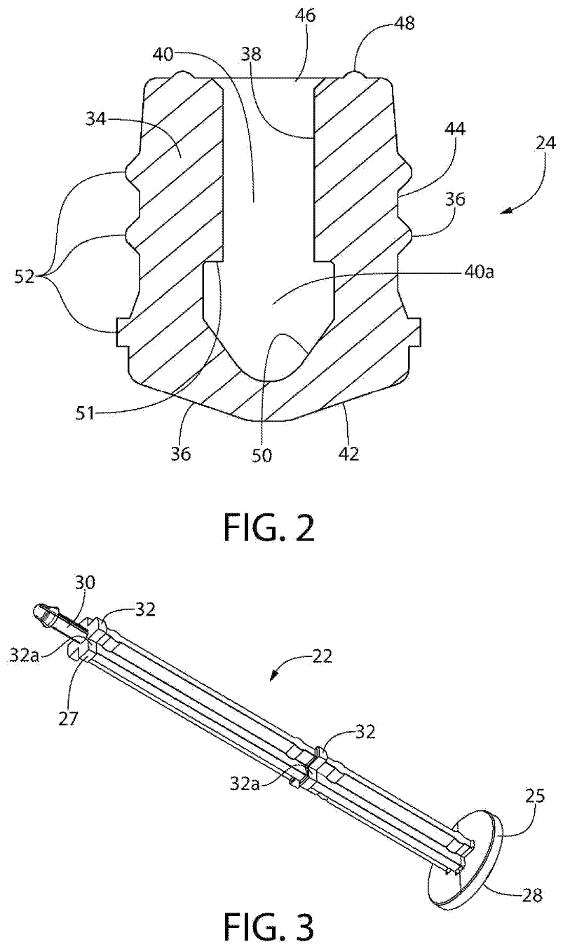

An exemplary plunger 24 usable in accordance with aspects of the disclosed concept is shown in FIG. 2. The plunger 24 comprises a plunger sleeve 34 having an exterior surface 36 and an interior surface 38 surrounding an inner cavity 40. The exterior surface 36 comprises a distal nose cone 42 and an outer annular wall 44 extending proximally therefrom. The outer annular wall 44 may include one or more ribs 52 and leads to an opening 46 at a proximal end 48 of the plunger sleeve 34. The opening 46 is configured to receive the axial protrusion 30 as discussed above, such that the axial protrusion 30 is capable of extending into the inner cavity 40 and optionally contacts an engagement surface 50 of the interior surface 38. The engagement surface 50 is configured to receive a force applied in a distal direction by the plunger rod 22 to move the plunger assembly 20 in a distal direction. The inner cavity 40 can include a distal compartment 40a having a wider internal geometry than that of the portion of the inner cavity 40 (e.g., a cylindrical portion) leading up to the distal compartment 40a. The proximal end of the distal compartment 40a defines an annular and linear ledge 51 configured to catch or engage a shelf of a head portion of a plunger rod axial protrusion, as discussed below.

When assembled with the plunger rod 22 and disposed within a medical barrel 12, the plunger 24 is configured to provide sufficient compressive force against the inner wall 14 of a prefilled syringe or cartridge barrel to effectively seal and preserve the shelf-life of the contents of the barrel during storage. In embodiments in which the plunger 24 is a convertible plunger, the plunger 24 provides container closure integrity (CCI) and gas-tight scaling (e.g., providing a barrier to oxygen, moisture and/or optionally additional gases), adequate to effectively seal and preserve the shelf-life of the contents of the barrel during storage, when the plunger is in an “expanded state” or “storage mode.” The expanded state or storage mode may be a product of, for example, an expanded outer diameter or profile of at least a portion of the syringic barrel-contacting surface of the plunger and/or the normal force that the plunger 24 exerts on the inner wall of the syringe barrel in which it is disposed. The plunger 24 (or at least a portion of its exterior surface 36) is optionally reducible and/or reconfigurable to what may alternatively be characterized as a “constricted state” or a “dispensing mode,” wherein the compressive force against the sidewall of the barrel is reduced or eliminated in part, allowing a user to more easily advance the plunger 24 in the barrel 12 and thus dispense the contents of the syringe or cartridge. As discussed in greater detail below, conversion from storage mode to dispensing mode is effectuated by elongation of the plunger 24. Prior to elongation, the plunger 24 may be said to be in its natural state or “pre-elongation state.” When the plunger 24 is disposed within a medical barrel, the pre-elongation state is synonymous with the expanded state or storage mode.

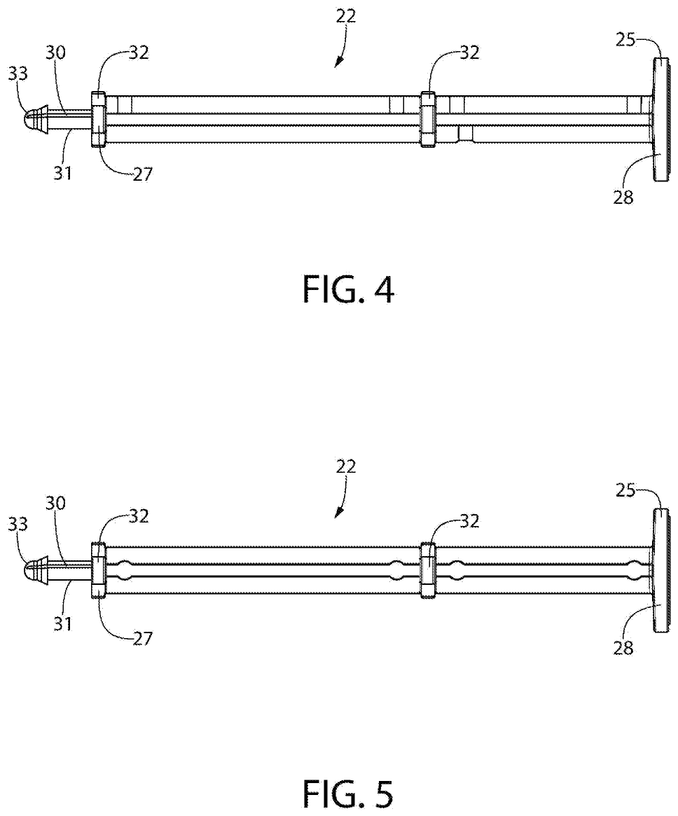

Referring to FIGS. 3-7, in one optional embodiment, the plunger rod 22 and the axial protrusion 30 are of unitary construction. “Unitary construction” can imply or mean a single manufactured piece or an assembly in which the assembled components (e.g., plunger rod and axial protrusion) are rigidly secured to each other or integrally formed so as to move together in any direction as a unitary part. The plunger rod 22 is an elongate member having a proximal end 25 and distal end 27. The plunger rod 22 can optionally include a disc shaped thumb rest 28 at the proximal end 25. The axial protrusion 30 can be secured to (and in this case, integral with) and can extend outwardly from the distal end 27 of the plunger rod 22.

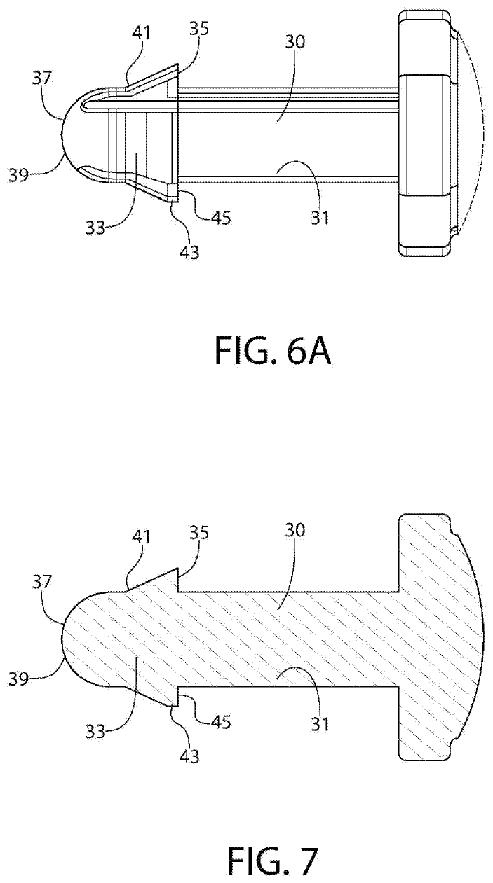

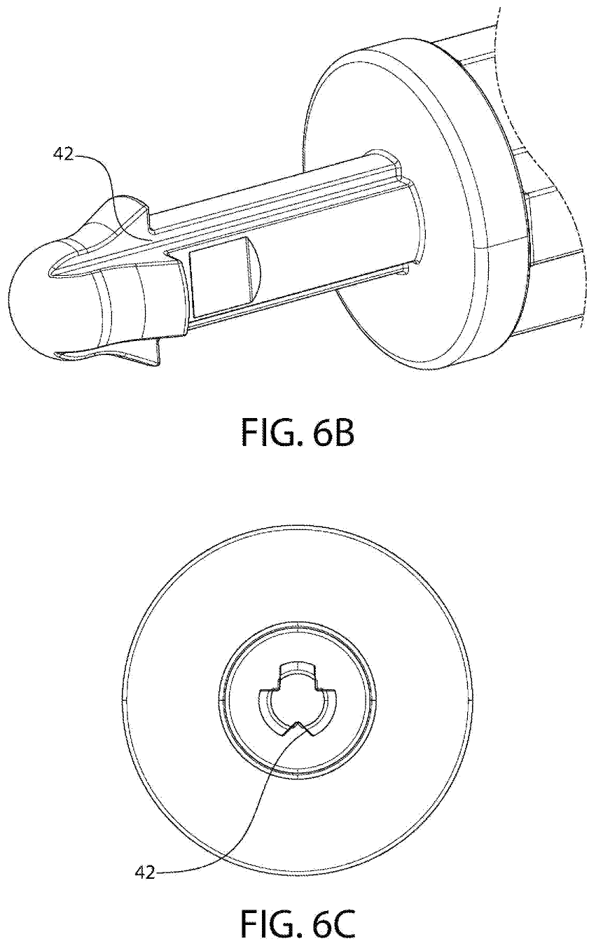

As shown in FIGS. 6A-6C and 7, at least a portion the axial protrusion 30 can be generally cylindrical and of essentially or exactly uniform cross section about a stem portion 31 thereof. The stem portion 31 of the axial protrusion 30 can lead to a head portion 33. The head portion 33 is optionally bell-shaped, as shown in the figures. At least the proximal end 35 of the head portion 33 has a greater cross-sectional width or diameter than that of the stem portion 31 and/or the distal end 37 of the head portion 33. The distal end 37 of the head portion 33 optionally includes a rounded tip 39.

In one optional embodiment, the head portion 33 can have a symmetrical cross-section that flares radially outward in a proximal direction, optionally along a curved surface 41 (e.g., having a radius with an imaginary center located outside of the head portion 33) and terminates at an outer edge 43 at the proximal end 35 of the head portion 33. Extending radially inward from the outer edge 43 is a linear and annular shelf 45 of the head portion 33 that terminates at the stem portion 21.

Referring to FIGS. 6A-6C, in one optional embodiment, one or both of the stem portion 21 and/or the head portion 33 can include at least one slot or groove 42. The slot 42 can extend parallel to the longitudinal axis of the stem portion 21 and/or the plunger rod 22. The slot 42 can also extend inwardly into the stem portion 21 and/or the head portion 33 from the outer peripheral surface thereof toward a central longitudinal axis of the stem portion 21 and/or the plunger rod 22. The slot 42 can be beneficial during sterilization because it allows sterilizing gas to pass through or by the head portion 33 and/or the stem portion 21. Optionally, two, three, or more radially and/or evenly spaced-apart slots 42 can extend around the periphery of the stem portion 21 and/or the head portion 33.

The head portion 33 is configured to facilitate easy insertion of the axial protrusion 30 into the inner cavity 40 and the distal compartment 40a of the plunger 24. In one embodiment, this act of inserting the axial protrusion 30 into the inner cavity and the distal compartment 40a of the plunger 24, while the plunger 24 is disposed in a medical barrel of a prefilled syringe, does not cause the plunger 24 to advance in a distal direction. At the same time, in one embodiment, the head portion 33 couples to the plunger 24 via the inner cavity 40 in such a way as to prevent the plunger rod 22 and plunger 24 from being decoupled too easily and/or inadvertently. Each of these functional considerations tend to be competing, so it is an object of the disclosed concept to strike the right balance to achieve these ends. The presently disclosed technology was found to strike this balance, which was challenging because it required simultaneously meeting contradicting needs, namely easy insertion but still being able to retract the plunger in the barrel.

In an optional embodiment that has demonstrated an ability to strike the right balance in a West 24324023/50 0.5 mL syringe stopper, the rounded tip 39 of the head portion 33 has a radius of 0.78 mm. At a point where the aforementioned radius of the rounded tip 39 ends, the head portion 33 has a diameter of about 1.55 mm. The curved surface 41 has a radius of about 1.00 mm and the axial distance from the beginning of the curved surface 41 to the shelf 45 is about 0.99 mm. The outward radial flare in the proximal direction of the head portion 33 is about 153° relative to the central axis of the head portion 33. Optionally, the outer edge 43 is rounded with a radius of about 0.05 mm.

The presently disclosed technology seeks to balance the following features or characteristics: loading, retraction, break loose force, and ethylene oxide sterilization. The loading is defined as the amount required to move on insertion. In one optional embodiment, the loading is ideally zero. Ideally the design does not add to the break loose force, while retaining the functionality of the plunger rod with the axial protrusion. Ethylene oxide sterilization is used to sterilize the plunger cavity. Applicant conducted testing to identify the options to achieve the optimum balance of the above characteristics.

One of the testing methods included filling the syringes with 0.165 mL of water and vacuum loading WEST™ FLUROTEC™ plungers. The water used was MILL1-Q™ high purity water. The plunger rods were stored for a predescribed amount of time at room temperature. The time period included 1 day, 3 or 4 days, 7 days, and 1 month, and ten samples per test group were tested at each time period.

Before commencement of a particular test, the plunger rod was suspended a few millimeters above the plunger. To determine loading force, the plunger rod was then compressed at a rate of 50 mm/min until reaching a displacement of 13 mm. In another test to prime and then retract the plunger rod, the plunger rod was retracted at a rate of 50 mm/min until reaching a displacement of 25 mm. In yet another test to measure the retraction force without priming, the plunger rod was retracted at a rate of 50 mm/min until reaching a displacement of 25 mm. Priming the plunger reduces the force required to move the plunger.

The design of the presently disclosed technology was tested against prior art device (i.e., a coupled, bayonet style rod) to measure loading force. At the 1 day testing period, it was determined that 3 out of 10 of the plunger rods of the presently disclosed technology moved. At the 4 day testing period, it was determined that 1 out of 10 of the plunger rods of the presently disclosed technology moved. At both the 7 day and 1 month testing periods, none of the plunger rods of the presently disclosed technology moved. In contrast to prior art devices, at each of the 1, 4, and 7 day testing periods all of the prior art devices moved. At the 1 month testing period, 8 out of 10 of the prior art devices moved.

The presently disclosed technology passed loading requirement after 7 days at room temperature (equivalent to about 21 days at 4° C.). Prior art coupled plunger rods that are intended for plunger retraction did not meet the plunger loading objective.

Break loose forces increase as the syringe ages (e.g., as storage time increases), so the force to insert the design of the presently disclosed technology is less than what is needed to move the plunger after several days. Ideally, plunger movement should be eliminated at later time points.

One type of prior art device is an uncoupled plunger rod, which will always retract without moving the plunger. Another type of prior art device is a traditional plunger rod (e.g., see FIGS. 2, 3A, and 3B of WO 2019199901), which will always move the plunger when retracting before pulling out of the plunger One of the problems that a prior art uncoupled plunger rod solves is the movement of the plunger during insertion that occurs with a traditional plunger rod, which can cause loss of seal integrity. The prior art uncoupled plunger rod teaches away form a coupled plunger rod to overcome this issue.

In ophthalmic applications and some other prefilled syringes, it is not desirable to move the plunger backward or away from the distal end of the medical barrel. Therefore, a syringe that needs to be primed first before use can be an advantage. Otherwise, the plunger rod could pull out without moving the plunger. Moving the plunger to prime the plunger starts the injection sequence.

When priming and then measuring the retention force, the design of the presently disclosed technology performed equally as well as the prior art devices (i.e., a coupled, bayonet style plunger rod). At each of the 1 day, 4, day, 7 day, and 1 month testing periods, 10 out of 10 of the design of the presently disclosed technology and the prior art device (i.e., a coupled, bayonet style plunger rod) retracted. In the presently disclosed technology, the plunger 24 moves backwards before the force is reached where the plunger mcd 22 pulls out of the plunger 24.

Applicant studied the effect of plunger rod design on break loose force. In this test, the syringes were filled with 0.165 ml of water. WEST™ FLUROTEC™ plungers were vacuum loaded. The plunger rods were then immediately loaded into the plungers. The devices were stored for 7 days at 40° C. The syringes were then allowed to reach room temperature, and the OVS cap was removed. The syringe and plunger rod were loaded into the test fixture, and the plunger force was tested at 190 mm/min. Applicant's tests revealed that there is no increase in the break loose force with the design of the presently disclosed technology.

Applicant's test also revealed that the removal force of the design of the presently disclosed technology is greater than the average glide force at the 2 year point. The retention force needed for the design of the presently disclosed technology is higher without first priming the syringe, as described above. These tests revealed that the presently disclosed technology strikes a good balance of loading and but still being able to retract the plunger 22 in the medical barrel 12.

Applicant measured the loading force with drug-filled syringes for the design of the presently disclosed technology, the prior art plunger rod without coupling features, and the prior art plunger rod with traditional coupling features. Applicant's testing demonstrated that after 7 days, the plunger of the presently disclosed technology does not move on insertion, which demonstrates that the presently disclosed technology achieves the optimum balance discussed above.

Applicant primed the drug filled syringes prior to retraction for the presently disclosed technology, prior art plunger rod without coupling features, and prior art plunger rod with traditional coupling features (e.g., see FIGS. 2, 3A, and 3B of WO 2019199901). The prior art plunger rod without coupling features did not retract the plungers at all. The design of the presently disclosed technology and the prior art plunger rod with traditional coupling features both passed 100% of the time.

When performing ISO 7886-1 Annex D, Item D.3.4 testing (300 kPA for 30 sec) with drug-filled syringes, the design of the presently disclosed technology, the prior art plunger rod without coupling features, and the prior art plunger rod with traditional coupling features all passed.

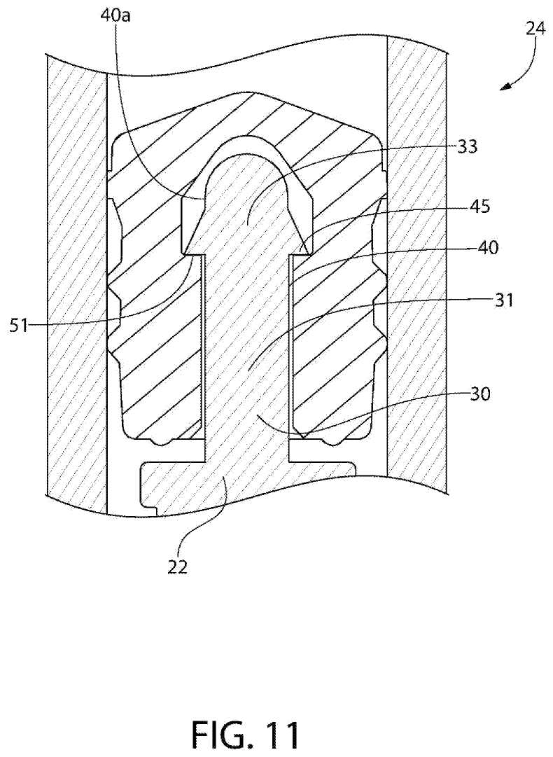

FIG. 11 shows an optional embodiment in which the head portion 33 couples to the plunger 24 via mating geometry such that the shelf 45 is retained, such as by engaging or mating contact, by the ledge 51. When retained, the shelf 45 and the ledge 51 can extend in parallel and can be in contact with one another.

As shown in FIGS. 3-5, the plunger rod 22 and/or a shaft thereof can optionally include one or more radial stabilizing members 32, which may loosely engage or sightly contact the inner wall of a medical barrel 12 when in use, to stabilize the plunger rod 22 (e.g., by preventing wobbling), as the plunger assembly 20 is advanced down the barrel 12. The stabilizing members 32 optionally include notches 32a therein to enable flow of gas therethrough during gas sterilization of the plunger rod 22.

As discussed above, the plunger 24, as part of a plunger assembly 20, is configured to be disposed within the medical barrel 12 of the syringe, such as a prefilled syringe. In that position, when sufficient distal force is applied to the plunger assembly 20, the plunger 24 is advanced down the medical barrel 12 to dispense the injectable liquid 16 from the dispensing end 13 of the medical barrel 12, e.g., through a needle. When this occurs, the plunger 24 (if optionally in a convertible configuration) is converted from storage mode to dispensing mode. In storage mode, the plunger 24 provides a tight seal, as set forth above. This tight seal may provide a level of radial compression against the inner wall 14 of the medical barrel 12 that makes it difficult to advance the plunger 24 down or with respect to the barrel 12. When a user initially applies sufficient distal force onto the plunger assembly 20, the plunger 24 optionally begins to stretch axially, causing at least a portion of the outer annular wall 44 of the plunger sleeve 34 to constrict slightly to at least slightly reduce the radial compression against the inner wall 14, while still providing a liquid seal, thus providing a more desirable glide force than would be achievable without elongating the plunger sleeve 34.

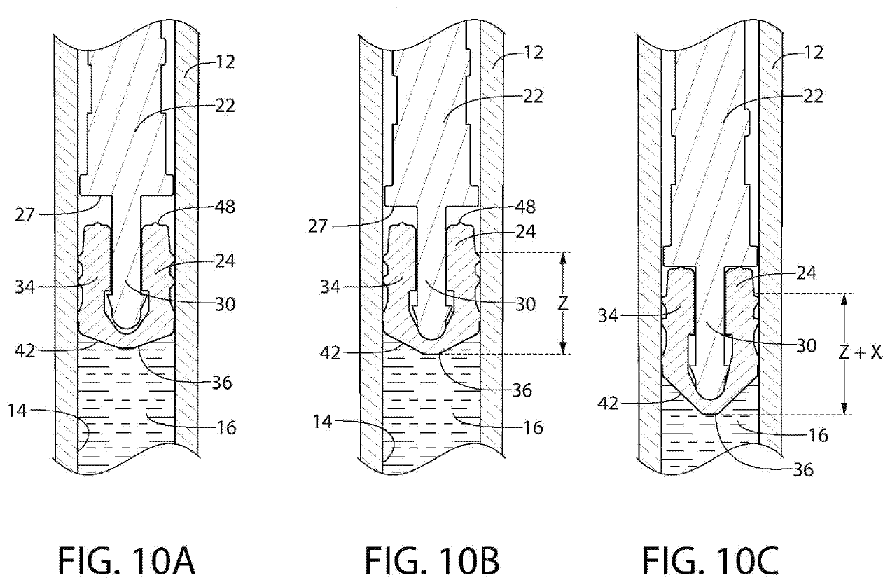

Referring now to FIGS. 10A-10C, when the plunger 24 is in the form of a convertible plunger, conversion of the plunger 24 of plunger assembly 20 from storage mode to dispensing mode is illustrated. FIG. 10A shows the plunger 24 in the pre-elongation state or storage mode. In this position, the axial protrusion 30 applies little or no distal force to the engagement surface 50 of the interior surface 38 of the plunger sleeve 34 (see FIG. 2). Optionally, the distal end 37 of the heat portion 33 does not contact the engagement surface 50 in the pre-elongation state or storage mode. As such, the plunger 24 is in its “fattest” or widest state, providing its greatest radial compression or force against the inner wall 14 of the medical barrel 12. e.g., to provide CCI over shelf life of the product 12. In this configuration, the diameter or cross-sectional width of the axial protrusion 30 preferably does provide radial support to reinforce or add the seal that the plunger 24 provides. However, in this embodiment and in the configuration shown in FIG. 10A, the axial protrusion 30 can be relatively loosely fitted within the inner cavity 40 of the plunger sleeve 34, without an interference fit.

FIGS. 10B and 10C illustrate transition of the plunger 24 to dispensing mode. When a user applies sufficient initial distal force onto the plunger rod 22, this causes the axial protrusion 30 to move into contact with and apply force in a distal direction onto the engagement surface 50 of the plunger (see FIG. 2). Optionally, a portion of the plunger sleeve 34 may initially adhere to the inner wall via “sticktion”. As this happens, the plunger 24 axially elongates along a stretch zone Z, causing the plunger 24 to slightly constrict (e.g., radially inward) about the stretch zone Z. Constriction of the plunger 24 reduces radial compression onto the sidewall 14 of the medical barrel 12, thus converting the plunger 24 into the dispensing mode. As the distal force continues or increases, the stretch zone increases to zone Z+X. The plunger 24 may thus be more easily advanced down the medical barrel 12, all the while maintaining a liquid tight seal and optionally CCI. In one optional embodiment, FIG. 10R shows a priming mode, and FIG. 10C shows the dispensing mode.

As explained above, the plunger sleeve 34 can optionally include a narrower section of the inner cavity 40 proximal to the distal compartment 40a. When the head portion 33 occupies the distal compartment 40a of the inner cavity 40, the axial protrusion 30 cannot be readily manually pulled out of the plunger 24 because the head portion 33 is of a greater diameter or cross-sectional width than the narrower section of the inner cavity 40. Therefore, pulling back (in a proximal direction) on the plunger rod 22 with a sufficient force will thus proximally displace the plunger 24.

Optionally, in any embodiment, the axial protrusion 30 is provided within the inner cavity 40 of the plunger sleeve 34 as the only component disposed therein. The axial protrusion 30 is not secured to the plunger 24 or to an insert within the plunger by a threaded engagement.

Optionally, in any embodiment, when the plunger 24 is in the storage position (e.g., see FIG. 10A), the distal end 27 of the plunger rod 22 does not contact (e.g., is spaced-apart from) the proximal end 48 of the plunger 24. Optionally, in some embodiments, when the plunger 24 is in the dispensing mode or position, the distal end 27 of the plunger rod 22 does not contact the proximal end of the plunger 24. In other words, optionally in both storage position (pre-elongation state) and dispensing position (after elongation), a space may be provided between the proximal end of the plunger and the distal end of the plunger rod. Optionally, in some embodiments, as shown in FIG. 10C, when the plunger 24 is in the dispensing mode or position, the distal end 27 of the plunger rod 27 contacts the proximal end 48 of the plunger 24.

Optionally, in any embodiment in which the axial protrusion 30 is of the same diameter along its length up until the head portion 33 thereof, the axial protrusion 30 is equal to or less than 1.8 mm in diameter, optionally equal to or less than 1.6 mm in diameter. Optionally, in any such embodiment, the axial protrusion 30 is from 1.45 mm to 1.8 mm in diameter, optionally from 1.45 mm to 1.6 mm in diameter. Optionally, where the axial protrusion 30 is of the same diameter along its entire length until the head portion 33 thereof), the diameter is such that it contacts the inner cavity 40 of the plunger sleeve 34 to reinforce the plunger's ability to provide a seal without being engaged in an interference fit with the inner cavity.

Optionally, in any embodiment, the syringe is a 0.5 mL syringe, as that term is understood in the industry.

Optionally, in any embodiment, the plunger is a West 2342 4023/50 plunger.

### PECVD Coating Layers

In another aspect, the presently disclosed technology optionally includes use of any embodiments (or combination of embodiments) of plungers according to the disclosed concept in syringes having a PECVD coating or PECVD coating set. The syringes may be made from, e.g., glass or plastic. Optionally, the syringe barrel according to any embodiment is made from an injection moldable thermoplastic material as defined above, in particular a material that appears clear and glass-like in final form. e.g., a cyclic olefin polymer (COP), cyclic olefin copolymer (COC) or polycarbonate. Such materials may be manufactured, e.g., by injection molding, to very tight and precise tolerances (generally much tighter than achievable with glass). This is a benefit when trying to balance the competing considerations of seal tightness and low plunger force in plunger design.

This section of the disclosure focuses primarily on prefilled syringes as one implementation of optional aspects of the presently disclosed technology. Again, however, the presently disclosed technology can include any parenteral container that utilizes a plunger, such as syringes that are empty, cartridges, auto-injectors, prefilled syringes, or prefilled cartridges.

For some applications, it may be desired to provide one or more coatings or layers to the interior wall of a parenteral container to modify the properties of that container. For example, one or more coatings or layers may be added to a parenteral container. e.g., to improve the barrier properties of the container and prevent interaction between the container wall (or an underlying coating) and drug product held within the container. Such coatings or layers may be constructed in accordance with the teachings of PCT Application PCT/US2014/023813, filed on Mar. 11, 2014, which is incorporated by reference herein in its entirety.

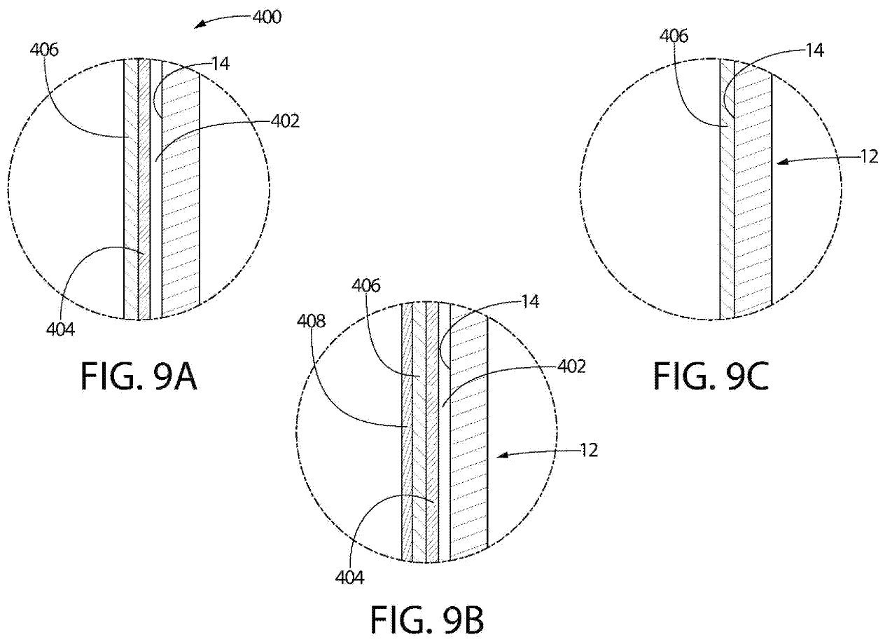

For example, as shown in FIG. 9A, which is a first alternative embodiment of an enlarged sectional view of the medical barrel 12 of the syringe assembly 10 of FIG. 8A, the inner surface 14 of the medical barrel 12 may include a coating set 400 comprising one or more coatings or layers. The medical barrel 12 may include at least one tie coating or layer 402, at least one barrier coating or layer 404, and at least one organo-siloxane coating or layer 406. The organo-siloxane coating 406 can have pH protective properties. This embodiment of the coating set 400 is referred to herein as a “tri-layer coating set” in which the barrier coating or layer 404 is protected against contents having a pH otherwise high enough to remove it by being sandwiched between the pH protective organo-siloxane coating 406 and the tie coating 402. The contemplated thicknesses of the respective layers in nanometers (preferred ranges in parentheses) are given in the following Tri-layer Thickness Table:

Tri-layer Thickness Table

| Adhesion (nm) | Barrier (nm) | Protection (nm) |
|---|---|---|
| 5-100 | 20-200 | 50-500 |
| (5-20) | (20-30) | (100-200) |

Properties and compositions of each of the coatings that make up the tri-layer coating set are now described.

The tie coating 402 has at least two functions. One function of the tie coating 402 is to improve adhesion of a barrier coating 404 to a substrate (e.g., the inner surface 14 of the barrel 12), in particular a thermoplastic substrate, although a tie layer can be used to improve adhesion to a glass substrate or to another coating or layer. For example, a tie coating, also referred to as an adhesion layer or coating, can be applied to the substrate and the barrier layer can be applied to the adhesion layer to improve adhesion of the barrier layer or coating to the substrate.

Another function of the tie coating 402 has been discovered: a tie coating 402 applied under a barrier coating 404 can improve the function of a pH protective organo-siloxane coating 406 applied over the barrier coating 404.

The tie coating 402 can be composed of, comprise, or consist essentially of SiOxCy, in which x is between 0.5 and 2.4 and y is between 0.6 and 3. Alternatively, the atomic ratio can be expressed as the formula SiwOxCy. The atomic ratios of Si, O, and C in the tie coating 402 are, as several options:
Si 100: O 50-150: C 90-200 (i.e. w=1, x=0.5 to 1.5, y=0.9 to 2);
Si 100: O 70-130: C 90-200 (i.e. w=1, x=0.7 to 1.3, y=0.9 to 2)
Si 100: O 80-120: C 90-150 (i.e. w=1, x=0.8 to 1.2, y=0.9 to 1.5)
Si 100: O 90-120: C 90-140 (i.e. w=1, x=0.9 to 1.2, y=0.9 to 1.4), or
Si 100: O 92-107: C 116-133 (i.e. w=1, x=0.92 to 1.07, y=1.16 to 1.33).

The atomic ratio can be determined by XPS. Taking into account the H atoms, which are not measured by XPS, the tie coating 402 may thus in one aspect have the formula SiwOxCyHz (or its equivalent SiOxCy), for example where w is 1, x is from about 0.5 to about 2.4, y is from about 0.6 to about 3, and z is from about 2 to about 9. Typically, a tie coating 402 would hence contain 36% to 41% carbon normalized to 100% carbon plus oxygen plus silicon.

The barrier coating 404 for any embodiment defined in this specification (unless otherwise specified in a particular instance) is a coating or layer, optionally applied by PECVD as indicated in U.S. Pat. No. 7,985,188, which is hereby incorporated by reference. The barrier coating preferably is characterized as a “SiOx” coating, in which x, the ratio of oxygen to silicon atoms, is from about 1.5 to about 2.9. The thickness of the SiOx or other barrier coating or layer can be measured, for example, by transmission electron microscopy (TEM), and its composition can be measured by X-ray photoelectron spectroscopy (XPS). The barrier layer is effective to prevent oxygen, carbon dioxide, water vapor, or other gases (e.g., residual monomers of the polymer from which the container wall is made) from entering the container and/or to prevent leaching of the pharmaceutical material into or through the container wall.

Preferred methods of applying the barrier layer 404 and tie layer 402 to the inner surface 14 of the barrel 12 is by plasma enhanced chemical vapor deposition (PECVD), such as described in. e.g., U.S. Pat. App. Pub. No. 20130291632, which is incorporated by reference herein in its entirety.

Barrier layers or coatings of SiOx are eroded or dissolved by some fluids, for example aqueous compositions having a pH above about 5. Since coatings applied by chemical vapor deposition can be very thin—tens to hundreds of nanometers thick—even a relatively slow rate of erosion can remove or reduce the effectiveness of the barrier layer in less time than the desired shelf life of a product package. This is particularly a problem for fluid pharmaceutical compositions, since many of them have a pH of roughly 7, or more broadly in the range of 5 to 9, similar to the pH of blood and other human or animal fluids. The higher the pH of the pharmaceutical preparation, the more quickly it erodes or dissolves the SiOx coating. Optionally, this problem can be addressed by protecting the barrier coating or layer, or other pH sensitive material, with a pH protective organo-siloxane coating or layer.

Optionally, the pH protective coating 406 can be composed of, comprise, or consist essentially of SiwOxCyHz (or its equivalent SiOxCy) or SiwNxCyHz or its equivalent SiNxCy). The atomic ratio of Si:O:C or Si:N:C can be determined by XPS (X-ray photoelectron spectroscopy). Taking into account the H atoms, the pH protective coating or layer may thus in one aspect have the formula SiwOxCyHz, or its equivalent SiOxCy, for example where w is 1, x is from about 0.5 to about 2.4, y is from about 0.6 to about 3, and z is from about 2 to about 9.

Typically, expressed as the formula SiwOxCy, the atomic ratios of Si, O, and C arc, as several options:
Si 100: O 50-150: C 90-200 (i.e. w=1, x=0.5 to 1.5, y=0.9 to 2);
Si 100: O 70-130: C 90-200 (i.e. w=1, x=0.7 to 1.3, y=0.9 to 2)
Si 100: O 80-120: C 90-150 (i.e. w=1, x=0.8 to 1.2, y=0.9 to 1.5)
Si 100: O 90-120: C 90-140 (i.e. w=1, x=0.9 to 1.2, y=0.9 to 1.4)
Si 100: O 92-107: C 116-133 (i.e. w=1, x=0.92 to 1.07, y=1.16 to 1.33),
or
Si 100: O 80-130: C 90-150.

Alternatively, the organo-siloxane coating or layer can have atomic concentrations normalized to 100% carbon, oxygen, and silicon, as determined by X-ray photoelectron spectroscopy (XPS) of less than 50% carbon and more than 25% silicon. Alternatively, the atomic concentrations are from 25 to 45% carbon, 25 to 65% silicon, and 10 to 35% oxygen. Alternatively, the atomic concentrations are from 30 to 40% carbon, 32 to 52% silicon, and 20 to 27% oxygen. Alternatively, the atomic concentrations are from 33 to 37% carbon, 37 to 47% silicon, and 22 to 26% oxygen.

Optionally, the atomic concentration of carbon in the pH protective coating or layer 406, normalized to 100% of carbon, oxygen, and silicon, as determined by X-ray photoelectron spectroscopy (XPS), can be greater than the atomic concentration of carbon in the atomic formula for the organosilicon precursor. For example, embodiments are contemplated in which the atomic concentration of carbon increases by from 1 to 80 atomic percent, alternatively from 10 to 70 atomic percent, alternatively from 20 to 60 atomic percent, alternatively from 30 to 50 atomic percent, alternatively from 35 to 45 atomic percent, alternatively from 37 to 41 atomic percent.

Optionally, the atomic ratio of carbon to oxygen in the pH protective coating or layer 406 can be increased in comparison to the organosilicon precursor, and/or the atomic ratio of oxygen to silicon can be decreased in comparison to the organosilicon precursor.

An exemplary empirical composition for a pH protective coating according to an optional embodiment is SiO_{1.3}C_{0.8}H_{3.6}.

Optionally in any embodiment, the pH protective coating 406 comprises, consists essentially of, or consists of PECVD applied coating.

Optionally in any embodiment, the pH protective coating 406 is applied by employing a precursor comprising, consisting essentially of, or consisting of a silane. Optionally in any embodiment, the silane precursor comprises, consists essentially of, or consists of any one or more of an acyclic or cyclic silane, optionally comprising, consisting essentially of, or consisting of any one or more of silane, trimethylsilane, tetramethylsilane. Si2-Si4 silanes, triethyl silane, tetraethyl silane, tetrapropylsilane, tetrabutylsilane, or octamethylcyclotetrasilane, or tetramethylcyclotetrasilane.

Optionally in any embodiment, the pH protective coating 406 comprises, consists essentially of, or consists of PECVD applied amorphous or diamond-like carbon. Optionally in any embodiment, the amorphous or diamond-like carbon is applied using a hydrocarbon precursor. Optionally in any embodiment, the hydrocarbon precursor comprises, consists essentially of, or consists of a linear, branched, or cyclic alkane, alkene, alkadiene, or alkyne that is saturated or unsaturated, for example acetylene, methane, ethane, ethylene, propane, propylene, n-butane, i-butane, butane, propyne, butyne, cyclopropane, cyclobutane, cyclohexane, cyclohexene, cyclopentadiene, or a combination of two or more of these. Optionally in any embodiment, the amorphous or diamond-like carbon coating has a hydrogen atomic percent of from 0.1% to 40%, alternatively from 0.5% to 10%, alternatively from 1% to 2%, alternatively from 1.1 to 1.8%

Optionally in any embodiment, the pH protective coating 406 comprises, consists essentially of, or consists of PECVD applied SiN. Optionally in any embodiment, the PECVD applied SiN is applied using a silane and a nitrogen-containing compound as precursors. Optionally in any embodiment, the silane is an acyclic or cyclic silane, optionally comprising, consisting essentially of, or consisting of silane, trimethylsilane, tetramethylsilane, Si2-Si4 silanes, triethylsilane, tetraethylsilane, tetrapropylsilane, tetrabutylsilane, octamethylcyclotetrasilane, or a combination of two or more of these. Optionally in any embodiment, the nitrogen-containing compound comprises, consists essentially of, or consists of any one or more of: nitrogen gas, nitrous oxide, ammonia or a silazane. Optionally in any embodiment, the silazane comprises, consists essentially of, or consists of a linear silazane, for example hexamethylene disilazane (HMDZ), a monocyclic silazane, a polycyclic silazane, a polysilsesquiazane, or a combination of two or more of these.

Optionally in any embodiment, the PECVD for the pH protective coating 406 is carried out in the substantial absence or complete absence of an oxidizing gas. Optionally in any embodiment, the PECVD for the pH protective coating or layer 406 is carried out in the substantial absence or complete absence of a carrier gas.

Optionally an FTIR absorbance spectrum of the pH protective coating 406 SiOxCyHz has a ratio greater than 0.75 between the maximum amplitude of the Si—O—Si symmetrical stretch peak normally located between about 1000 and 1040 cm−1, and the maximum amplitude of the Si—O—Si asymmetric stretch peak normally located between about 1060 and about 1100 cm−1. Alternatively in any embodiment, this ratio can be at least 0.8, or at least 0.9, or at least 1.0, or at least 1.1, or at least 1.2. Alternatively in any embodiment, this ratio can be at most 1.7, or at most 1.6, or at most 1.5, or at most 1.4, or at most 1.3. Any minimum ratio stated here can be combined with any maximum ratio stated here, as an alternative embodiment.

Optionally, in any embodiment the pH protective coating 406, in the absence of the liquid filling, has a non-oily appearance. This appearance has been observed in some instances to distinguish an effective pH protective coating or layer 406 from a lubricity layer (e.g., as described in U.S. Pat. No. 7,985,188), which in some instances has been observed to have an oily (i.e., shiny) appearance.

The pH protective coating 406 optionally can be applied by plasma enhanced chemical vapor deposition (PECVD) of a precursor feed comprising an acyclic siloxane, a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors. Some particular, non-limiting precursors contemplated for such use include octamethylcyclotetrasiloxane (OMCTS).

Other precursors and methods can be used to apply the pH protective coating 406 or passivating treatment. For example, hexamethylene disilazane (HMDZ) can be used as the precursor. HMDZ has the advantage of containing no oxygen in its molecular structure. This passivation treatment is contemplated to be a surface treatment of the SiOx barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH₃ and bonding of S—(CH₃)₃ to the silicon (it is contemplated that hydrogen atoms will be evolved and bond with nitrogen from the HMDZ to produce NH₃).

Another way of applying the pH protective coating 406 is to apply the pH protective coating 406 as an amorphous carbon or fluorocarbon coating, or a combination of the two.

Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings can be derived from fluorocarbons (for example, hexafluoroethylene or tetrafluoroethylene). Either type of coating, or a combination of both, can be deposited by vacuum PECVD or atmospheric pressure PECVD. It is contemplated that an amorphous carbon and/or fluorocarbon coating will provide better passivation of an SiOx barrier layer than a siloxane coating since an amorphous carbon and/or fluorocarbon coating will not contain silanol bonds.

It is further contemplated that fluorosilicon precursors can be used to provide a pH protective coating or layer over a SiOx barrier layer. This can be carried out by using as a precursor a fluorinated silane precursor such as hexafluorosilane and a PECVD process. The resulting coating would also be expected to be a non-wetting coating.

Yet another coating modality contemplated for protecting or passivating a SiOx barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 60 and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resin on a SiOx barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting a SiOx layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide® can be used to provide a pH protective coating or layer 406 that also provides lubricity.

Thus, a pH protective coating fora thermoplastic syringe wall according to an aspect of the presently disclosed technology may comprise, consist essentially of, or consist of any one of the following; plasma enhanced chemical vapor deposition (PECVD) applied coating having the formula SiOxCyHz, in which x is from 0 to 0.5, alternatively from 0 to 0.49, alternatively from 0 to 0.25 as measured by X ray photoelectron spectroscopy (XPS), y is from about 0.5 to about 1.5, alternatively from about 0.8 to about 1.2, alternatively about 1, as measured by XPS, and z is from 0 to 2 as measured by Rutherford Backscattering Spectrometry (RBS), alternatively by Hydrogen Forward Scattering Spectrometry (HFS); or PECVD applied amorphous or diamond-like carbon, CHz, in which z is from 0 to 0.7, alternatively from 0.005 to 0.1, alternatively from 0.01 to 0.02; or PECVD applied SiNb, in which b is from about 0.5 to about 2.1, alternatively from about 0.9 to about 1.6, alternatively from about 1.2 to about 1.4, as measured by XPS.

PECVD apparatus suitable for applying any of the PECVD coatings or layers described in this specification, including the tie coating or layer, the barrier coating or layer or the organo-siloxane coating or layer, is shown and described in U.S. Pat. No. 7,985,188 and U.S. Pat. App. Pub. No. 20130291632. This apparatus optionally includes a vessel holder, an inner electrode, an outer electrode, and a power supply. A vessel seated on the vessel holder defines a plasma reaction chamber, optionally serving as its own vacuum chamber. Optionally, a source of vacuum, a reactant gas source, a gas feed or a combination of two or more of these can be supplied. Optionally, a gas drain, not necessarily including a source of vacuum, is provided to transfer gas to or from the interior of a vessel seated on the port to define a closed chamber.

It is contemplated that syringes having a plunger-contacting inner surface are provided substantially without the presence of a flowable lubricant. As used herein, “substantially without the presence of a flowable lubricant,” means that a flowable lubricant (e.g., PDMS) is not provided to a syringe barrel in amounts that would contribute to the lubricity of the plunger-syringe system. Since it is sometimes the practice to use a flowable lubricant when handling plungers prior to assembling them into syringes. “substantially without the presence of a flowable lubricant” in some cases may contemplate the presence of trace amounts of such lubricant as a result of such handling practices.

Accordingly, in one optional aspect, the presently disclosed technology may incorporate an organo-siloxane coating on the inner surface of a parenteral container, which provides lubricious properties conducive to acceptable plunger operation. The organo-siloxane coating may, for example, be any embodiment of the pH protective coating discussed above. The organo-siloxane coating may be applied directly to the interior wall of the container or as a top layer on a multi-layer coating set, e.g., the tri-layer coating set discussed above.

The organo-siloxane coating can optionally provide multiple functions: (1) a pH resistant layer that protects an underlying layer or underlying polymer substrate from drug products having a pH from 4-10, optionally from 5-9; (2) a drug contact surface that minimizes aggregation, extractables and leaching; (3) in the case of a protein-based drug, reduced protein binding on the container surface; and (4) a lubricating layer, e.g., to facilitate plunger advancement when dispensing contents of a syringe.

Use of an organo-siloxane coating on a polymer-based container as the contact surface for a plunger provides distinct advantages. Plastic syringes and cartridges may be injection molded to tighter tolerances than their glass counterparts. It is contemplated that the dimensional precision achievable through injection molding allows optimization of the inside diameter of a syringe to provide sufficient compression to the plunger for CCI and gas-tightness on the one hand, while not over-compressing the plunger so as to provide desired plunger force upon administration of the drug product. Optimally, this would eliminate or dramatically reduce the need for lubricating the syringe or cartridge with a flowable lubricant.

Lubricity coatings, e.g., prepared according to methods disclosed in U.S. Pat. No. 7,985,188 (incorporated by reference herein in its entirety), are particularly well suited to provide a desired level of lubricity for plungers in parenteral containers. Such lubricity coatings are preferably applied using plasma enhanced chemical vapor deposition (“PECVD”) and may have one of the following atomic ratios. SiwOxCy or SiwNxCy, where w is 1, x is from about 0.5 to 2.4 and y is from about 0.6 to about 3. Such lubricity coatings may have a thickness between 10 and 500 nm. Advantages of such plasma coated lubricity layers may include lower migratory potential to move into the drug product or patient than liquid, sprayed or micron-coated silicones. It is contemplated that use of such lubricity coatings to reduce plunger force is within the broad scope of the presently disclosed technology. Optionally, as shown in FIG. 9B, a PECVD lubricity coating 408 may be disposed on top of a tri-layer coating set, making a four layer coating set.

The PECVD coating apparatus and process are as described generally in PECVD protocols of U.S. Pat. No. 7,985,188, or PCT/US16/47622, which are incorporated here by reference.

In one embodiment, the tie or adhesion coating or layer and the barrier coating or layer, and optionally the pH protective layer, are applied in the same apparatus, without breaking vacuum between the application of the adhesion coating or layer and the barrier coating or layer or, optionally, between the barrier coating or layer and the pH protective coating or layer. During the process, a partial vacuum is drawn in the lumen. While maintaining the partial vacuum unbroken in the lumen, a tie coating or layer of SiOxCy is applied by a tie PECVD coating process. The tie PECVD coating process is carried out by applying sufficient power to generate plasma within the lumen while feeding a gas suitable for forming the coating. The gas feed includes a linear siloxane precursor, optionally oxygen, and optionally an inert gas diluent. The values of x and y are as determined by X-ray photoclectron spectroscopy (XPS). Then, while maintaining the partial vacuum unbroken in the lumen, the plasma is extinguished. A tie coating or layer of SiOxCy, for which x is from about 0.5 to about 2.4 and y is from about 0.6 to about 3, is produced on the inside surface as a result.

Later during the process, while maintaining the partial vacuum unbroken in the lumen, a barrier coating or layer is applied by a barrier PECVD coating process. The barrier PECVD coating process is carried out by applying sufficient power to generate plasma within the lumen while feeding a gas. The gas feed includes a linear siloxane precursor and oxygen. A barrier coating or layer of SiOx, wherein x is from 1.5 to 2.9 as determined by XPS is produced between the tie coating or layer and the lumen as a result.

Then, optionally, while maintaining the partial vacuum unbroken in the lumen, the plasma is extinguished.

Later, as a further option, a pH protective coating or layer of SiOxCy can be applied. In this formula as well, x is from about 0.5 to about 2.4 and y is from about 0.6 to about 3, each as determined by XPS. The pH protective coating or layer is optionally applied between the barrier coating or layer and the lumen, by a pH protective PECVD coating process. This process includes applying sufficient power to generate plasma within the lumen while feeding a gas including a linear siloxane precursor, optionally oxygen, and optionally an inert gas diluent.

Then, optionally, while maintaining the partial vacuum unbroken in the lumen, the plasma is extinguished.

Later, as a further option, a lubricity coating or layer of SiOxCy can be applied. In this formula as well, x is from about 0.5 to about 2.4 and y is from about 0.6 to about 3, each as determined by XPS. The lubricity coating or layer is optionally applied on top of the pH protective coating, by a lubricity PECVD coating process. This process includes applying sufficient power to generate plasma within the lumen while feeding a gas including an organo siloxane precursor, optionally oxygen, and optionally an inert gas diluent.

Optionally in any embodiment, the PECVD process for applying the tie coating or layer, the barrier coating or layer, and/or the pH protective coating or layer, and/or the lubricity coating or any combination of two or more of these, is carried out by applying pulsed power (alternatively the same concept is referred to in this specification as “energy”) to generate plasma within the lumen.

Alternatively, the tie PECVD coating process, or the barrier PECVD coating process, or the pH protective PECVD coating process, or any combination of two or more of these, can be carried out by applying continuous power to generate plasma within the lumen.

Trilayer Coating Process Protocol (all Layers Coated in the Same Apparatus)

The trilayer coating as described in this embodiment is applied by adjusting the flows of a single organosilicon monomer (HMDSO) and oxygen and also varying the PECVD generating power between each layer (without breaking vacuum between any two layers).

The vessel (e.g., a COC syringe) is placed on a vessel holder, sealed, and a vacuum is pulled within the vessel. After pulling vacuum, the gas feed of precursor, oxygen, and argon is introduced, then at the end of the “plasma delay” continuous (i.e., not pulsed) RF power at 13.56 MHz is turned on to form the tie coating or layer. Then power is turned off, gas flows are adjusted, and after the plasma delay power is turned on for the second layer—an SiOx barrier coating or layer. This is then repeated for a third layer before the gases are cut off, the vacuum seal is broken, and the vessel is removed from the vessel holder. The layers are put down in the order of Tie then Barrier then pH Protective. An exemplary process settings are as shown in the following table:

| | O₂ | | HMDSO | Power | Deposition |
|---|---|---|---|---|---|
| Coating | (sccm) | Ar (sccm) | (sccm) | (W) | Time (sec) |
| Tie | 1 | 40 | 2 | 20 | 2.5 |
| Barrier | 100 | 0 | 1 | 60 | 15 |
| pH Protective | 1 | 40 | 2 | 20 | 10 |

As a still further alternative, pulsed power can be used for some steps, and continuous power can be used for others. For example, when preparing a trilayer coating or layer composed of a tie coating or layer, a barrier coating or layer, and a pH protective coating or layer, an option specifically contemplated for the tie PECVD coating process and for the pH protective PECVD coating process is pulsed power, and an option contemplated for the corresponding barrier layer is using continuous power to generate plasma within the lumen.

### Optional Injectable Product Compositions

Optionally, in any embodiment, syringes according to the disclosed concept are prefilled with an injectable drug product.

Optionally in any embodiment, the injectable drug product may be an ophthalmic drug suitable for intravitreal injection. Optionally in any embodiment, the ophthalmic drug includes a VEGF antagonist, optionally an anti-VEGF antibody or an antigen-binding fragment of such antibody. Optionally in any embodiment, the VEGF antagonist comprises Ranibizumab.

Aflibercept, or a combination of these.

Optionally in any embodiment, the concentration of the liquid formulation of an ophthalmic drug suitable for intravitreal injection is 1 to 100 mg of the drug active agent per ml, of the liquid formulation 40 (mg/ml), alternatively 2-75 mg/ml, alternatively 3-50 mg/ml, alternatively 5 to 30 mg/ml and alternatively 6 or 10 mg/ml.

Optionally in any embodiment, the liquid formulation of an ophthalmic drug suitable for intravitreal injection comprises 6 mg/mL, alternatively 10 mg/mL, of Ranibizumab.

Optionally in any embodiment, the ophthalmic drug suitable for intravitreal injection further comprises: a buffer in an amount effective to provide a pH of the liquid formulation 40 in the range from about 5 to about 7; a non-ionic surfactant in the range of 0.005 to 0.02% mg/mL of complete formulation, alternatively in the range of 0.007 to 0.018% mg/mL of complete formulation, alternatively in the range of 0.008 to 0.015% mg/mL of complete formulation, alternatively in the range of 0.009 to 0.012% mg./mL of complete formulation, alternatively in the range of 0.009 to 0.011% mg./mL of complete formulation, alternatively 0.01% mg./mL of complete formulation; and water for injection.

Optionally in any embodiment, the ophthalmic drug suitable for intravitreal injection comprises 6 mg/mL, or thereabouts, of Ranibizumab: 100 mg/mL of α,α-trehalose dihydrate, 1.98 mg/mL L-histidine; and 0.1 mg/mL Polysorbate 20 in water for injection.

### Preparation and Priming of Prefilled Syringe for Use

In optional embodiments, for example in ophthalmic applications, the administrator (e.g., healthcare provider) may wish to or be required to prime a prefilled syringe according to the disclosed concept, before use. The prefilled syringe may be filled with excess drug product beyond the needed dose. A dose line may be provided on the syringe, indicative of the dose to be administered to the patient. To prime the syringe, a user may hold the syringe with the needle side facing up to allow any air bubbles to rise to the top (i.e., the distal end of the syringe). Next, the user may expel any air by pushing the plunger rod in a distal direction until the edge of the nose cone of the plunger is aligned with the dose mark so that the precise dose is ready to be administered to the patient, e.g., into the patient's eye tissue.

When priming the syringe, initiation of movement of the plunger requires that the user apply the break loose force, as discussed above. Once the plunger has been advanced in the priming step and the break loose force has been applied, movement of the plunger thenceforth would require application of glide force, which is lower than break loose force. For example, break loose force may be about 8-10 N while glide force may be about 1-4 N. In an optional aspect, the prefilled syringe and plunger assembly is configured to allow retraction of the plunger rod to overcome the glide force but not the break loose force.

Prior to priming, retraction may separate the plunger rod from the plunger, depending on the type of plunger rod used since the retention force is less than the break loose force. For example, for a traditional plunger rod, there would be no separation. For a prior art uncoupled plunger rod, there would be separation. For the presently disclosed technology, separation would often or usually occur. After priming, the plunger rod of the presently disclosed technology is able to retract the plunger in the medical barrel because the retention force is greater than the glide force.

In an optional aspect, therefore, the disclosed concept uniquely provides a plunger rod that can couple to the plunger while the plunger is disposed in the prefilled syringe and not separate from the plunger when retracting the plunger rod, thereby overcoming the glide force after a priming step.

### Industry Standards for Testing Aspects of Plunger

Testing of compression setting properties of the plunger assembly may be conducted using methods known in the art, for example, ASTM D395.

Testing of adhesive properties or bonding strength between a film (e.g., fluoropolymer) and the plunger may be conducted using methods known in the art, for example, according to ASTM D1995-92(2011) or D1876-08.

Plunger sliding force is the force required to maintain movement of a plunger in a syringe or cartridge barrel, for example during aspiration or dispense. It can advantageously be determined using, e.g., the ISO 7886-1:1993 test known in the art, or the test method to be incorporated into ISO 11040-4. Plunger breakout force, which may be tested using the same method as that for testing plunger sliding force, is the force required to start a stationary plunger moving within a syringe or cartridge barrel. Machinery useful in testing plunger sliding and breakout force is, e.g., an Instron machine using a 50 N transducer.

Testing for extractables. i.e., amount of material that migrates from the plunger into the liquid within the syringe or cartridge, may be conducted using methods set forth in Ph. Eur. 2.9.17 Test for Extractable Volume of Parenteral Preparations, for example.

Testing of container closure integrity (CCI) may be done using a vacuum decay leak detection method, wherein a vacuum is maintained inside of a test volume and pressure rise is measured over time. A large enough pressure rise is an indication that there is flow into the system, which is evidence of a leak. Optionally, the vacuum decay test is implemented over two separate cycles. The first cycle is dedicated to detecting large leaks over a very short duration. A relatively weak vacuum is pulled for the first cycle because if a gross leak is detected, a large pressure differential is not necessary to detect a large pressure rise. Use of a first cycle as described helps to shorten total test time if a gross leak exists. If no leak is detected in the first cycle, a second cycle is run, which complies with ASTM F2338-09 Standard Test Method for Nondestructive Detection of Leaks in Packages by Vacuum Decay Method. The second cycle starts out with a system evaluation to lower the signal to noise ratio in the pressure rise measurements. A relatively strong vacuum is pulled for a long period of time in the second cycle to increase the chance of detecting a pressure rise in the system.

Testing of air leakage past the syringe piston during aspiration may be conducted using methods known in the art, for example, ISO 7886-1:1993.

Testing of liquid leakage at a syringe piston under compression may be conducted using methods known in the art, for example, ISO 7886-1:2015, Annex B for liquid leakage, with blocked fluid path, by applying an axial force on the plunger stopper by final plunger rod, consistent with the maximum force generated during use.

In an exemplary method of applying this standard, a 0.5 mL syringe may be filled with 0.165 mL MILLI-Q high purity water. Plungers, optionally West FLUROTEC plungers are vacuum loaded into the filled syringes. Plunger assemblies with axial protrusions are disposed within the plungers as described in this specification to place plungers into storage mode. The crosshead compresses at a rate of 10 mm/min until reaching a maximum force of 5.43 N, which corresponds to 300 kPa pressure in the syringe (or a force consistent with the maximum force generated during use). The crosshead makes small adjustments to hold at the maximum force for 30 seconds. In this implementation, the ISO 7886-1 test is considered failed if any water from inside the syringe moves back past any rib on the plunger.

Various aspects of the invention will be illustrated in more detail with reference to the following Examples, but it should be understood that the present invention is not deemed to be limited thereto.

### EXAMPLES

### Example 1

### Insertion and Retention of Axial Protrusion In Plunger

Data showing performance of plunger rod embodiments according to aspects of the disclosed concept are provided herein.

The following exemplary embodiments further describe optional aspects of the presently disclosed technology and are part of this Detailed Description. These exemplary embodiments are set forth in a format substantially akin to claims (each with numerical designations followed by a letter), although they are not technically claims of the present application. The following exemplary embodiments refer to each other in dependent relationships as “embodiments” instead of “claims.”

1A. A device used in conjunction with a medical barrel for delivering liquid drug formulation, the device comprising:
a thumb rest at a proximal end of a shaft;
an axial protrusion extending distally from a flat or planar distal end of the shaft, the axial protrusion having a stem portion having a cylindrical shape of uniform cross section, a head portion located at a distal end of the stem portion, the head portion having a proximal end with a greater cross-sectional width or diameter than that of the stem portion, wherein at least one of the stem portion and the head portion including at least one slot extending parallel to a longitudinal axis of the shaft, the slot being configured to allow sterilizing gas to pass therethrough.

2A. The device of embodiment 1A, wherein the axial protrusion includes three radially spaced-apart slots.

3A. The device of embodiment 1A or 2A, wherein the head portion has a distal end comprising a rounded tip, the head portion having a symmetrical cross-section with an outer contour that flares radially outward in a proximal direction.

4A. The device of any one of embodiment 1A-3A, wherein the axial protrusion is configured to engage a stopper.

5A. The device of embodiment 4A, wherein the combined device and stopper are configured to be inserted into a medical barrel.

1B. A method of using a prefilled syringe, the prefilled syringe including a plunger assembly and a medical barrel, the plunger assembly including a plunger rod, an axial protrusion secured to or extending from a planar distal end of the plunger rod, and a plunger configured to receive at least a portion of the axial protrusion, the axial protrusion including a head portion having a proximal end with a greater cross-sectional width or diameter than that of a stem portion thereof, at least one of the stem portion and the head portion including at least one slot extending parallel to a longitudinal axis of the shaft, the slot being configured to allow sterilizing gas to pass therethrough, the method comprising:
advancing the plunger down the medical barrel to dispense a portion of a liquid drug formulation in a priming step; and
inserting a needle into a patient.

2B. The method of embodiment 1B, wherein the head portion has a symmetrical cross-section with an outer contour that flares radially outward in a proximal direction.

3A. A method of using a prefilled syringe, the prefilled syringe including a plunger assembly and a medical barrel, the plunger assembly including a plunger rod, an axial protrusion secured to or extending from a planar distal end of the plunger rod, and a plunger configured to receive at least a portion of the axial protrusion, the method comprising:
priming the plunger rod to at least slightly stretch the plunger; and
inserting a needle into a patient after the plunger has been primed.

3B. The method of embodiment 3A, wherein the axial protrusion includes a head portion having a proximal end with a greater cross-sectional width or diameter than that of a stem portion thereof.

3C. The method of embodiment 3A or 3B, wherein at least one of the stem portion and the head portion includes at least one slot extending parallel to a longitudinal axis of the shaft, the slot being configured to allow sterilizing gas to pass therethrough.

3D. The method of embodiment 3A, 3B, or 3C. wherein the head portion has a symmetrical cross-section with an outer contour that flares radially outward in a proximal direction, at least a portion of the outer contour optionally having a curved surface comprising a radius with an imaginary center located outside of the head portion.

3E. The method of embodiment 3A, 3B, 3C, or 3D, wherein the plunger is not moved proximally.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the present presently disclosed technology.

1. A plunger assembly for use in a medical barrel, comprising:
a plunger rod having a distal end and a proximal end;
an axial protrusion secured to or extending from the distal end of the plunger rod, the axial protrusion comprising a stem portion having an essentially uniform cross section, the stem portion leading to a head portion, the head portion having a proximal end with a greater cross-sectional width or diameter than that of the stem portion, the head portion having a distal end comprising a rounded tip, the head portion having a symmetrical cross-section with an outer contour that flares radially outward in a proximal direction, at least a portion of the outer contour having a curved surface comprising a radius with an imaginary center located outside of the head portion, the outer contour terminating at an outer edge at the proximal end of the head portion, the head portion comprising a shelf extending radially inward from the outer edge and terminating at the stem portion; and
a plunger comprising a plunger sleeve having an exterior surface and an interior surface surrounding an inner cavity, the exterior surface comprising a distal nose cone and an outer annular wall extending proximally from the nose cone and leading to an opening at a proximal end of the plunger sleeve, the opening receiving the axial protrusion such that the axial protrusion extends into the inner cavity and contacts an engagement surface of the interior surface, the engagement surface configured to receive a force applied in a distal direction by the axial protrusion to move the plunger assembly in a distal direction when the plunger rod is moved in a distal direction;
wherein the distal end of the plunger rod does not initially contact the proximal end of the plunger sleeve when the plunger is in a pre-elongation state, and
wherein the plunger rod and axial protrusion are provided as a single piece of unitary construction.

2. The plunger assembly of claim 1, the inner cavity of the plunger comprising a distal compartment having a wider internal geometry than that of a portion of the inner cavity leading up to the distal compartment, the distal compartment having a proximal end that defines a ledge configured to catch or engage the shelf of the head portion to enable the head portion to couple to the plunger.

3. A pre-filled syringe comprising:
a medical barrel having an inner wall and product containing area, the product containing area having disposed therein an injectable product, for example a liquid composition, the medical barrel having a distal dispensing end for dispensing the injectable product and an open proximal end configured for receipt of a plunger assembly; and
the plunger assembly according to claim 1, wherein the plunger is disposed within the medical barrel such that the nose cone faces the injectable product and at least a portion of the plunger rod extends proximally from the open proximal end of the medical barrel.

4. The pre-filled syringe of claim 3, wherein application of sufficient force onto the plunger rod in a distal direction causes the plunger assembly to displace distally down the medical barrel.

5. The pre-filled syringe of claim 4, the plunger comprising a stretch zone that is configured to undergo elongation along a central axis of the plunger upon application of a force in the distal direction by the axial protrusion onto the engagement surface, wherein the elongation reduces an outer profile of the outer annular wall along the stretch zone.

6. The pre-filled syringe of claim 5, wherein the plunger rod and axial protrusion are configured such that the plunger rod does not contact the proximal end of the plunger sleeve when the assembly is advanced in a distal direction down the medical barrel, so as not to axially compress the plunger during actuation.

7. The pre-filled syringe of claim 6, wherein application of axial force in a proximal direction onto the proximal end of the plunger rod sufficient to axially displace the plunger rod in a proximal direction over a predetermined distance does not disconnect or remove the axial protrusion from the inner cavity.

8. (canceled)

9. (canceled)

10. The pre-filled syringe of claim 3, wherein the inner wall of the medical barrel comprises a plasma enhanced chemical vapor deposition (PECVD) coating or coating set.

11. The prefilled syringe of claim 10, wherein the PECVD coating or coating set is selected from the group consisting of:
a bilayer coating set, comprising a tie layer and a SiOx barrier layer disposed on the tie layer;
a trilayer coating set, comprising a tie layer, an SiOx barrier layer disposed on the tie layer and an organo-siloxane layer disposed on the SiOx barrier layer; and
a four layer coating set, comprising a tie layer, an SiOx barrier layer disposed on the tie layer, an organo-siloxane layer disposed on the SiOx barrier layer and a lubricity layer disposed on the organo-siloxane layer.

12. (canceled)

13. The prefilled syringe of claim 3, wherein the prefilled syringe is a 0.5 mL syringe and the injectable product is a liquid ophthalmic drug formulation.

14. The prefilled syringe of claim 13, wherein the liquid ophthalmic drug formulation is suitable for intravitreal injection and comprises a VEGF antagonist, wherein the VEGF antagonist comprises an anti-VEGF antibody or an antigen-binding fragment of such antibody.

15. The prefilled syringe of claim 13, wherein the liquid ophthalmic drug formulation comprises Ranibizumab and/or Aflibercept.

16. (canceled)

17. A method of using a prefilled syringe, the prefilled syringe including a plunger assembly and a medical barrel, the plunger assembly including a plunger rod, an axial protrusion secured to or extending from a planar distal end of the plunger rod, and a plunger configured to receive at least a portion of the axial protrusion, the axial protrusion including a head portion having a proximal end with a greater cross-sectional width or diameter than that of a stem portion thereof, the head portion having a symmetrical cross-section with an outer contour that flares radially outward in a proximal direction, the method comprising:
advancing the plunger down the medical barrel to dispense a portion of a liquid drug formulation in a priming step; and
inserting a needle into a patient.

18. The method of claim 17, wherein the plunger assembly includes a first configuration and a second configuration, in the first configuration a head portion of the axial protrusion is located in a distal portion of an internal cavity of the plunger and the planar distal end of the plunger rod is spaced-apart from a proximal end of the plunger, in the second configuration the head portion of the axial protrusion is located in the distal portion of the internal cavity of the plunger and the planar distal end of the plunger rod contacts the proximal end of the plunger.

19. The method of claim 17, wherein the head portion comprises a symmetrical cross-section that flares radially outward from a distal end thereof toward a proximal end thereof along a curved surface.

20. The method of claim 17, wherein the plunger assembly includes a first configuration and a second configuration, in the first configuration a head portion of the axial protrusion is located in a distal portion of an internal cavity of the plunger and the planar distal end of the plunger rod is spaced-apart from a proximal end of the plunger, in the second configuration the head portion of the axial protrusion is located in the distal portion of the internal cavity of the plunger and the planar distal end of the plunger rod is spaced-apart from the proximal end of the plunger.

21. (canceled)

22. The method of claim 18, wherein an outer periphery of the plunger is reduced in the second configuration as compared to the first configuration.

23. (canceled)

24. The method of claim 23, wherein the step of inserting the needle into the patient comprises inserted in the needle into the patient's eye.

25. A device used in conjunction with medical barrel for delivering liquid drug formulation, the device comprising:
a thumb rest at a proximal end of a shaft;
an axial protrusion extending distally from a flat or planar distal end of the shaft, the axial protrusion having a stem portion having a cylindrical shape of uniform cross section, a head portion located at a distal end of the stem portion, the head portion having a proximal end with a greater cross-sectional width or diameter than that of the stem portion, the head portion having a distal end comprising a rounded tip, the head portion having a symmetrical cross-section with an outer contour that flares radially outward in a proximal direction.

26. The device of claim 25, wherein the outer contour of the head portion terminates at an outer edge at the proximal end of the head portion.

27. The device of claim 25, wherein the head portion comprises a shelf extending radially inward from the outer edge and terminating at the stem portion.

28. The device of claim 25, wherein at least a portion of the outer contour of the head portion having a curved surface comprising a radius with an imaginary center located outside of the head portion.

29. The device of claim 25, wherein the head portion is integrally formed with the axial protrusion to form a one-piece construction.

30. The device of claim 25, wherein the head portion is fixed with respect to the axial protrusion.

31. The device of claim 25, wherein at least one of the stem portion and the head portion includes at least one slot extending parallel to a longitudinal axis of the shaft, the slot being configured to allow sterilizing gas to pass therethrough.

32. The device of claim 31, wherein the at least one slot includes three radially spaced-apart slots.
